(19) United States
(12) Patent Application Publication
(10) Pub. No.: US 2006/0204464 A1
(43) Pub. Date: Sep. 14, 2006

(54) Macrocyclic thiiranes

(76) Inventor: Luca Turin, London (GB)

Correspondence Address: Dennis P. Clarke;Miles & Stockbridge P.C., Suite 500, 1751 Pinnacle Drive, McLean, VA, 22102-3833, US

(21) Appl. No.: 11/364,553

(22) Filed: Mar. 1, 2006

Related U.S. Application Data
(60) Provisional application No. 60/657,425, filed on Mar. 2, 2005.

(51) Int. Cl. A61K 8/49; C07D 331/02; C11D 3/50

(52) U.S. Cl. 424/70.1; 510/101; 549/1

(57) ABSTRACT

The present invention provides a compound having the formulae:

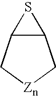

wherein Z is CH₂, CH═CH or CHSCH (thiirane), provided that if two or more Z are CH═CH they are not conjugated and wherein n is selected so that the total number of carbon atoms is from 8 to 20. Mixtures of these compounds, methods for their preparation, their use as perfume materials for application to a variety of substrates and their use in flavoring and in articles of manufacture is also provided.

The present invention relates generally to the field of flavors and fragrances. More particularly, the present invention relates to novel aromachemicals. These aromachemicals find utility in any and all applications requiring certain flavors and fragrances. The invention also provides mixtures of these aromachemicals, methods for their preparation and their use as perfume materials for application to a variety of substrates and their use in flavoring and articles of manufacture including the derivatives.

There are a large number and variety of known flavors and fragrances used as ingredients in perfumes and in a varied range of other products. For example, perfumes for application in laundry detergents, fabric softeners, rinse conditioners and other products intended for use on textile fibers primarily contain fragrances.

It is an object of the invention to provide novel aromachemical compounds. The present invention provides compounds of formula (I):

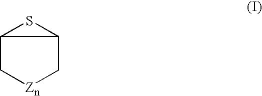

Wherein Z is CH₂, CH═CH or CHSCH (thiirane), provided that if two or more of Z are CH═CH they are not conjugated and wherein n is selected so that the total number of carbon atoms is from 8 to 20.

Preferably the compound of formula (I) comprises from 10 to 16 carbon atoms, for example 11, 12, 13, 14 or 15 and, most preferably, 12 carbons.

The compounds of the invention comprise from 0 to 5 CH═CH groups, preferably from 0 to 4, for example 0, 1, 2 or 3. The CH═CH groups are not conjugated.

The compounds of the invention comprise from I to 6 CHSCH (thiirane) groups (including the one shown in the formula above), preferably from 1 to 5, for example 1, 2, 3 or 4.

Preferably each Z is CH₂ and n is from 4 to 16, especially from 6 to 12, most preferably 8. These compounds can be represented by the formula (Ia): 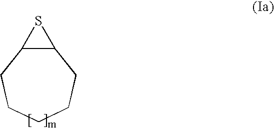
wherein m is from 2 to 14, preferably from 4 to 10, most preferably 6.

A specific example of a compound of the above formula is:

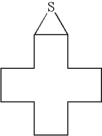

This compound possesses an earthy, woody, fresh odor.

The compounds of the invention contain one macrocyclic ring and one or more sulfur containing three membered rings (thiiurane).

The present invention also provides a method for synthesizing the compounds of the above formula. The compounds of the invention can be prepared from the “parent” cycloolefin and/or epoxide compounds but do not need to be prepared in this manner. That is, the compounds of the invention can be derived from synthetic strategies that do not involve the “parent” compounds.

The compounds of the above formulae may be prepared by reacting a compound of formula (II): 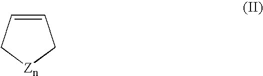
wherein Z and n are as defined above with a peroxy acid to produce an epoxide. Any suitable peroxy acid may be used. Suitable peroxy acids include peroxyacetic acid, peroxybenzoic and haloperbenzoic acids, for example m-chloroperbenzoic acid.

The compound of formula (II) may comprise more that one double bond provided that the double bonds are not conjugated. Preferred compounds of formula (II) for use in this reaction contain 1, 2, 3 or 4 double bonds. Most preferably, the compounds of formula (II) contain a single double bond.

The resulting epoxide of formula 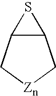
can be converted into the thiiurane by any suitable method. For example, the epoxide may be treated with a reagent comprising KSCN/silica gel to give the macrocyclic thiirane.

This method of producing the compounds of formula (Ia) is summarized in the following reaction scheme:

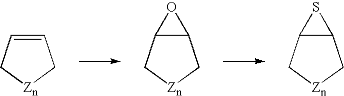

It will be appreciated that some epoxide compounds of the formula shown above are commercially available. Thus, the present invention also provides a process for producing a thiirane compound of formula (I) from the epoxide without first producing the epoxide from the parent alkene, i.e., a process that involves only the second step of the reaction scheme defined above.

It will be understood by those skilled in the art that, where necessary, the intermediate epoxides may be prepared from the unsaturated hydrocarbons according to any well-known conventional method. It will be further understood by those skilled in the art that the epoxides may be converted to the corresponding thiiuranes according to well known methods, such as, e.g., the method described in Chan et al, JAm. Chern. Soc., 1972, 94, 2880 [see also, Johnson et al, Tetrahedron Letters, vol. 38, no. 33, pp 5873-5876 (1997)].

The method for producing the preferred compounds of formula (Ia) can be summarized as follows:

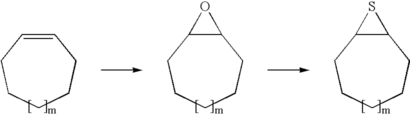

Again, it will be appreciated that some epoxide compounds of the formula shown above are commercially available. Thus, the present invention also provides a process for producing a thiirane compound of formula (Ia) from the epoxide without first producing the epoxide from the parent alkene, i.e. a process that involves only the second step of the reaction scheme defined above.

More particularly, the method for producing the compound of formula 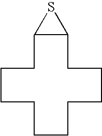
can be summarized as follows:

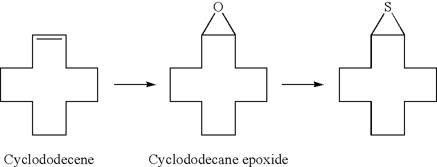

Cyclododecene and cyclododecane epoxide are both commercially available and can be obtained by suppliers such as Aldrich (UK). Thus, the present invention also provides a process for producing the thiirane compound starting from commercially obtained cyclododecane epoxide, i.e., a process that involves only the second step of this reaction scheme.

Cyclododecene can be obtained from commercial sources, alternatively it may be produced from 1,5,9-cyclododecatriene (shown below), which is also available commercially.

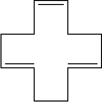

The present invention is predicated on the realization that the compounds of the invention possess potentially useful flavorant/odor profiles.

An advantage of replacing a CH═CH double bond with a thiiurane is that this produces a molecule with a higher molecular weight. The greater molecular weight can lower the volatility of the molecule, thereby potentially providing a middle note rather a top note, or a drydown note rather than a middle note.

The present invention provides for the use of the compounds of the invention and mixtures thereof as a flavor and/or fragrance.

The present invention also provides compositions, products, preparations or articles having improved aroma, fragrance or odor characteristics containing a compound or mixture of compounds of the invention as described above. The present invention also provides methods to confer, improve, enhance or modify the taste or flavor property of a composition, product, preparation or article which comprises adding thereto a flavor effective amount of a composition or mixture of compounds of the invention as described above.

A method to confer, improve, enhance or modify the aroma, fragrance or odor characteristics of compositions, products, preparations or articles which comprises adding thereto an aroma, fragrance or odor effective amount of a composition or mixture of compounds of the invention as described above is also provided.

The compounds of the invention can be included in virtually any article of manufacture that can include the non-derivatized fragrance or flavorant compound, or for that matter, other fragrances, whether natural or artificial. Examples include bleach, detergents, flavorings and fragrances, beverages, including alcoholic beverages, and the like. The compounds of the invention can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes.

The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds of the invention can be used as perfuming ingredients, as single compounds or as mixtures thereof, preferably in an amount of at least about 30% by weight of the perfume composition, more preferably in an amount of at least about 60% by weight of the composition. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the compounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products mentioned above.

The compounds may be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These aspects are described in more detail below.

In addition to the compounds of the invention, the compositions herein may include a detersive surfactant and optionally, one or more additional detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random CIO-20 alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, blotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of the invention herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of the invention can also be prepared. The compounds of the invention can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions of the invention may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds of the invention to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of the invention are then added to the mixer and mixed for a suitable amount of time. The gum can be then removed from the mixer and rolled to stick thickness on waxed paper while warm.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention is illustrated by the following non-limiting example.

### EXAMPLE 1

To cyclododecene in a suitable solvent (e.g., CHCl₃) was added a peroxy acid, m-chloroperbenzoic acid. The resulting epoxide (cyclododecane epoxide) thus formed was treated with a reagent comprising KSCN/silica gel [Brinmeyer et al, J. Org. Chern., 1980, 45, 4254-4255] to give the macrocyclic thiirane, cyclododecane thiirane.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A compound having the formula: 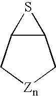
wherein Z is CH₂, CH═CH or CHSCH (thiirane), provided that if two or more of Z
are CH═CH they are not conjugated and wherein n is selected so that the total number of carbon atoms is from 8 to 20.

2. A compound according to claim 1 having the following formula: 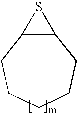
wherein m is from 2 to 14.

3. The compound according to claim 2 wherein m is 6.

4. A method of producing a compound of formula (I) as defined in claim 1, the method comprising converting the epoxide group of a compound having the formula: 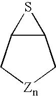
to the corresponding thiirane using any suitable reagent, wherein Z and n are as defined in claim 1.

5. The method of claim 4, comprising reacting a compound of the formula: 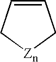
with a peroxy acid to produce the compound of formula:

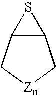

6. A method of producing a compound of claim 2, the method comprising converting the epoxide group of a compound having the formula: 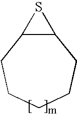
to the corresponding thiilrane using any suitable reagent, wherein m is as defined in claim 2.

7. The method of claim 6, comprising reacting a compound of formula 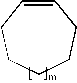
with a peroxy acid to produce the compound of formula

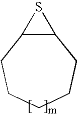

8. The method of claim 6 or 7, wherein m is 6.

9. The use of a compound or a mixture of compounds as defined in claim 1 as a flavor or fragrance.

10. A substrate treated with a compound or a mixture of compounds as defined in claim 1.

11. A method for treating a substrate to impart flavorant/fragrance releasing characteristics thereto comprising treating the substrate with a compound or a mixture of compounds as defined in claim 1.

12. A composition, product, preparation or article having aroma, fragrance or odor releasing characteristics containing a compound or mixture of compounds as defined in claim 1, optionally in admixture with other perfuming ingredients, solvents, or adjuvants of current use in the art.

13. A composition, product, preparation or article according to claim 12, wherein the compound or mixture of compounds as defined in claim is present in an amount of at least 30 percent by weight.

14. A composition, product, preparation or article according to claim 13, wherein the compound or mixture of compounds is present in an amount of at least 60 percent by weight.

15. A composition, product, preparation or article according to claim 12 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

16. A detergent composition, product, preparation or article according to claim 15, wherein the compound or mixture of compounds is in admixture with other detergent ingredients, solvents or adjuvants.

17. A bleach composition, product, preparation or article according to claim 15, wherein the compound or mixture of compounds is in admixture with other bleach ingredients, solvents or adjuvants.

18. A disinfectant composition, product, preparation or article according claim 15, wherein the compound or mixture of. compounds is in admixture with other disinfectant ingredients, solvents or adjuvants.

19. A composition, product, preparation or article according to claim 15 in the form of a body odorant, deodorant or antiperspirant wherein the compound or mixture of compounds is in admixture with other body odorant, deodorant or antiperspirant ingredients, solvents or adjuvants.

20. A composition, product, preparation or article having improved flavor or taste characteristics containing a compound or mixture of compounds as defined in claim 1.

21. A composition, product, preparation or article according to claim 20 in the form of a beverage, which optionally contains other beverage ingredients, solvents or adjuvants.

22. A composition, product, preparation or article according to claim 20 in the form of a flavoring, which optionally contains other flavoring ingredients, solvents or adjuvants.

23. A composition, product, preparation or article according to claim 20 in the form of a food, which optionally contains other food ingredients, solvents or adjuvants.

24. A composition, product, preparation or article according to claim 20 in the form of a chewing gum, which optionally contains other chewing gum ingredients, solvents or adjuvants.

25. A composition, product, preparation or article according to claim 20 in the form of a pharmaceutical, which optionally contains other pharmaceutical ingredients, solvents or adjuvants.

26. A composition, product, preparation or article according to claim 20 in the form of an orally-deliverable matrix material which may optionally contain other matrix material ingredients, solvents or adjuvants.

27. A method to confer, improve, enhance or modify a taste or flavor property of a composition, product, preparation or article which comprises adding thereto a flavor effective amount of a compound or mixture of compounds as defined in claim 1.

28. A method according to claim 27 wherein said composition, product, preparation or article is in the form of a beverage, a flavoring, a food, a chewing gum, a pharmaceutical or an orally deliverable matrix.

29. A method to confer, improve, enhance or modify an aroma, fragrance or odor characteristics of a composition, product, preparation or article which comprises adding thereto an aroma, fragrance or odor effective amount of a compound or mixture of compounds as defined in claim 1.

30. A method according to claim 29 wherein said composition, product, preparation or article is in the form of a perfume, a body odorant, deodorant or antiperspirant, a detergent, a bleach product or a disinfectant.

31. An article of manufacture comprising packaging material and an aroma, odor, fragrance, taste or flavor enhancing agent contained within the packaging material, wherein the agent is effective for the enhancement of the aroma, odor, fragrance, taste or flavor of a composition, preparation, product or article to which it is added, and wherein the packaging material comprises a label which indicates that the agent can be used for enhancing aroma, odor, fragrance, taste or flavor, and wherein the agent is a compound or mixture of compounds as defined in claim 1.
